(19) United States
(12) Patent Application Publication
(10) Pub. No.: US 2019/0010509 A1
(43) Pub. Date: Jan. 10, 2019

(54) DNA MOLECULE ENCODING 5'UTR THAT ENABLES HIGH-LEVEL EXPRESSION OF RECOMBINANT PROTEIN IN PLANT

(71) Applicant: National University Corporation Nara Institute of Science and Technology, Ikoma-shi, Nara (JP)

(72) Inventor: Ko KATO, Ikoma-shi, Nara (JP)

(21) Appl. No.: 15/569,960

(22) PCT Filed: Apr. 21, 2016
(86) PCT No.: PCT/JP2016/062679

§ 371 Date: Oct. 27, 2017

(30) Foreign Application Priority Data
Apr. 30, 2015 (JP) 2015-093062

(51) Int. Cl. C12N 15/82

(52) U.S. Cl. CPC C12N 15/8216

(57) ABSTRACT

The present invention addresses the problem of developing a DNA molecule encoding a 5′UTR that enables the high-level expression of a recombinant protein in a plant, and providing a technique for producing a recombinant protein efficiently. A nucleic acid construct composed of a polynucleotide encoding a protein and a 5′UTR comprising a nucleotide sequence represented by SEQ ID NO: 1 or 2 or a variant of the 5′UTR, wherein the 5′UTR is ligated to the polynucleotide. By using the nucleic acid construct, it becomes possible to efficiently produce a recombinant protein in a plant.

### TECHNICAL FIELD

The present invention relates to a DNA molecule encoding 5′UTR that enables high-level expression of a recombinant protein in a plant. The present invention also relates to a nucleic acid construct in which the DNA molecule is linked to a polynucleotide encoding a protein, an expression vector including the nucleic acid construct, a transformant having the expression vector, and a method for producing a recombinant protein using the transformant.

### BACKGROUND ART

Conventionally, a technology for introducing foreign genes into plants has been established, and high-level expression systems for this have been constructed, whereby production of recombinant proteins using plants is actively underway. However, the production of recombinant proteins using plants is not fully satisfactory from the standpoint of production efficiency, and therefore further improvement is desired.

It is known that, in gene expression in plants, which is broadly divided into transcription process and translation process, the initiation reaction of translation serves as rate-controlling reaction for production of protein (Non-Patent Document 1). The translation process is initiated in the following manner: a translation initiation factor is coupled to the cap structure located at 5′ end of mRNA, to allow 40S subunit of ribosome to be recruited to a 5′ untranslated region (5′UTR). Since the recruit efficiency of ribosome to mRNA greatly affects the translation efficiency, the 5′UTR serving as the scaffolding thereof is a very important factor that defines the translation efficiency of mRNA.

There are cases where protein production in a plant decreases under environmental stress such as temperature, osmotic pressure, and salt concentration and nutrient starvation stress. It has been reported that the decrease in translation efficiency by 5′UTR is also responsible for the decrease in protein production under such stress. In recent years, 5′UTR with which translation is not repressed even under such stress has been found, and use of such 5′UTR for protein production in plants has been attempted (Patent Documents 1 and 2, for example). However, the 5′UTRs reported in Patent Documents 1 and 2 are meant for escaping translational repression under stress and not for increasing the production quantity of proteins itself under non-stress environments.

It is also known that the protein production in a plant greatly changes, not only with environmental stress and nutrition starvation stress, but also with growing stages and developmental stages. More specifically, while the translational state becomes worse along with growth and development in many mRNAs, there are still present mRNAs actively being translated. Conventionally, however, no examination has been made on 5′UTR capable of producing a protein with high efficiency by focusing on the translational states according to growing stages and developmental stages.

### PRIOR ART DOCUMENTS

### Non-Patent Document

Non-Patent Document 1: Gebauer, F. and Hentze, M. W., 2004, Molecular mechanisms of translational control, Nat. Rev. Mol. Cell Biol., 5:827-835

### Patent Documents

Patent Document 1: WO 2011/021666
Patent Document 2: WO 2013/031821

### SUMMARY OF THE INVENTION

### Problems to be Solved by the Invention

An object of the present invention is to provide a technology for developing a DNA molecule encoding 5′UTR that enables high-level expression of a recombinant protein in a plant and producing a recombinant protein efficiently.

### Means for Solving the Problem

In order to solve the above problem, the present inventor has analyzed the translational states of all mRNA species according to growing stages and developmental stages, such as seedlings, grown plants, young leaves (expanding leaves), and mature leaves (expanded leaves), using Arabidopsis, and found that mRNA including 5′UTR consisting of a base sequence represented by any of SEQ ID NOs: 1 to 4 is translated actively in all growing and developmental stages. The inventor has also found that, by use of a nucleic acid construct obtained by ligating such 5′UTR or its variant to a polynucleotide encoding a protein, it is possible to produce a recombinant protein efficiently. The present invention has been accomplished after further examinations performed repeatedly based on the above findings.

That is, the present invention provides the inventive modes set forth below.

1. A DNA molecule encoding 5′UTR, including a polynucleotide defined in any of (i) to (iii) below:

(i) a polynucleotide consisting of a base sequence represented by SEQ ID NO: 1 or 2;

(ii) a polynucleotide consisting of a base sequence in which one or several bases of the base sequence represented by SEQ ID NO: 1 or 2 are substituted, deleted, or added, the polynucleotide exhibiting 5′UTR activity equivalent to that of the polynucleotide having the base sequence represented by SEQ ID NO: 1 or 2; and

(iii) a polynucleotide hybridized with a DNA fragment consisting of a base sequence complementary to the base sequence represented by SEQ ID NO: 1 or 2 under a stringent condition, the polynucleotide exhibiting 5′UTR activity equivalent to that of the polynucleotide having the base sequence represented by SEQ ID NO: 1 or 2.

2. The DNA molecule according to item 1, including a polynucleotide consisting of a base sequence represented by any of SEQ ID NOs: 1 to 6.
3. A nucleic acid construct including the DNA molecule according to item 1 or 2 that is linked to a 5′ end side of a polynucleotide encoding a protein.
4. A vector including the nucleic acid construct according to item 3.
5. A method for producing a transformant including introducing the vector according to item 4 to a plant or a plant cell.
6. A transformant obtained by transforming a plant or a plant cell with the vector according to item 4.
7. A method for producing a recombinant protein including culturing or cultivating the transformant according to item 6.

### Advantages of the Invention

According to the present invention, in production of a recombinant protein using a plant, the translation efficiency by 5′UTR has been improved, and thus it is possible to produce a recombinant protein efficiently. Also, in production of a recombinant protein using a plant, the production efficiency of the recombinant protein usually tends to decrease as the growth and development of the plant advance. According to the present invention, it is expected possible to suppress decrease in translation efficiency and maintain the ability to produce the recombinant protein even at advanced growing and developmental stages of the plant.

### BRIEF DESCRIPTION OF THE DRAWINGS

FIG. 1 is a view showing a conceptual view of polysome/microarray analysis together with a calculation method of a PR value.

FIG. 2 shows transcription initiation sites and distribution ratios of candidate mRNAs high in polysome ratio (PR) value, in which each figure on the horizontal axis represents the number of bases from initiation codon AUG, and each figure on the vertical axis represents the relative ratio of each transcription initiation site to the total number of tags.

FIG. 3 is a view showing an outline of a test plasmid DNA used in a DNA transient expression experiment.

FIG. 4 is a view showing an outline of a control plasmid DNA used in the DNA transient expression experiment.

FIG. 5 is a view showing the results of the DNA transient expression experiment using Arabidopsis protoplasts.

FIG. 6 is a view showing an outline of a binary vector used in agroinfiltration.

FIG. 7 is a view showing an outline of a control binary vector used in agroinfiltration.

FIG. 8 is a view showing the results of evaluation of the translational ability of candidate 5′UTR by agroinfiltration using tobacco plant.

FIG. 9 shows the results of evaluation of the translational ability of candidate 5′UTR at growing stages using Arabidopsis plant.

FIG. 10 shows the results of evaluation of the translational ability of candidate 5′UTR at developmental stages using Arabidopsis plant.

### EMBODIMENTS OF THE INVENTION

The present invention will be described hereinafter in more detail. It is to be noted that the abbreviated indications of amino acids, peptides, base sequences, nucleic acids, etc. are according to the stipulations in IUPAC and IUB, “Guideline for preparation of descriptions etc., including base sequence or amino acid sequence” (edited by Japan Patent Office), and commonly used symbols in the art. In particular, DNA represents deoxyribonucleic acid. RNA represents ribonucleic acid, and mRNA represents messenger RNA.

Molecular biological manipulation such as gene manipulation may be performed using a known method as appropriate. For example, unless otherwise specified, it may be performed according to a method described in Molecular Cloning: A Laboratory Manual 3rd Edition (Cold Spring Harbor Laboratory Press), etc.

### (1) DNA Molecule Encoding 5′UTR

The DNA molecule of the present invention is a DNA molecule encoding 5′UTR, including a polynucleotide defined in any of (i) to (iii) below:

(i) a polynucleotide consisting of a base sequence represented by SEQ ID NO: 1 or 2;

(ii) a polynucleotide consisting of a base sequence in which one or several bases of the base sequence represented by SEQ ID NO: 1 or 2 are substituted, deleted, or added, the polynucleotide exhibiting 5′UTR activity equivalent to that of the polynucleotide having the base sequence represented by SEQ ID NO: 1 or 2; and

(iii) a polynucleotide hybridized with a DNA fragment consisting of a base sequence complementary to the base sequence represented by SEQ ID NO: 1 or 2 under a stringent condition, the polynucleotide exhibiting 5′UTR activity equivalent to that of the polynucleotide having the base sequence represented by SEQ ID NO: 1 or 2.

The polynucleotide having the base sequence represented by SEQ ID NO: 1, out of the polynucleotides in (i) above, is a DNA molecule encoding 5′UTR in At1g20440 gene of Arabidopsis. The other polynucleotide having the base sequence represented by SEQ ID NO: 2 in (i) above is a DNA molecule encoding 5′UTR in At1g06760 gene of Arabidopsis.

In the polynucleotide in (ii) above, the number of bases substituted, deleted, or added may be one or several. More specifically, it may be 1 to 15, preferably 1 to 10, more preferably 1 to 8, especially preferably 1 to 8, 1 to 7, 1 to 6, 1 to 5, 1 to 4, 1 to 3, 1 or 2, or 1.

In the polynucleotide in (iii) above, the “stringent condition” means a condition in which a pair of polynucleotides high in sequence similarity can be specifically hybridized. The pair of polynucleotides high in sequence similarity means polynucleotides having an identity of not less than 80%, for example, preferably not less than 90%, more preferably not less than 95%, especially preferably not less than 98%. The identity of a pair of polynucleotides may be calculated using Blast homology search software under default setting. A specific example of the “stringent condition” includes a condition in which hybridization is performed under a temperature condition of 42° C. using 5×SSC (83 mM NaCl, 83 mM sodium citrate).

In the polynucleotides in (ii) and (iii) above, “exhibiting 5′UTR activity equivalent to that of the polynucleotide having the base sequence represented by SEQ ID NO: 1 or 2” means that, when a recombinant protein is produced in a plant (including a plant cell) using the polynucleotide in (ii) as 5′UTR, the expression level is equivalent to that obtained when the polynucleotide having the base sequence represented by SEQ ID NO: 1 or 2 is used as 5′UTR. More specifically, it is indicated that, assuming that the expression level of a recombinant protein produced using the polynucleotide having the base sequence represented by SEQ ID NO: 1 or 2 as 5′UTR is 100%, the expression level of a recombinant protein should be not less than 80%, preferably 85 to 120%, more preferably 90 to 120%, especially preferably 95 to 120%, when the recombinant protein is produced using the polynucleotide in (ii) or (iii) as 5′UTR.

Preferred examples of the polynucleotides in (ii) and (iii) include a polynucleotide (SEQ ID NO: 3) in which one base C is added to the side of 5′ end of the base sequence represented by SEQ ID NO: 1 and a polynucleotide (SEQ ID NO: 4) in which two bases AC are added to the side of 5′ end of the base sequence represented by SEQ ID NO: 1.

Further, since it is known that a base sequence on the side of 3′ end of 5′UTR (i.e., an initiation codon-neighboring base sequence) affects the translation efficiency, a preferred example of the polynucleotides in (ii) and (iii) above includes a polynucleotide in which the first to fifth bases, preferably the first to third bases, more preferably the first to second bases, from the side of 3′ end of the base sequence represented by SEQ ID NO: 1 or 2 have been substituted by other bases. From the standpoint of further improving the production efficiency of a recombinant protein, specific examples of the polynucleotides in (ii) and (iii) in this mode include one consisting of a base sequence in which the 76th base C of the base sequence represented by SEQ ID NO: 2 is substituted by A (SEQ ID NO: 6), one consisting of a base sequence in which the 105th and 106th bases CT of the base sequence represented by SEQ ID NO: 1 are substituted by AG, one consisting of a base sequence in which the 106th and 107th bases CT of the base sequence represented by SEQ ID NO: 3 are substituted by AG (SEQ ID NO: 5), and one consisting of a base sequence in which the 107th and 108th bases CT of the base sequence represented by SEQ ID NO: 4 are substituted by AG.

The polynucleotide in (i) above may be obtained from Arabidopsis according to a known technique, but also be obtained by chemical synthesis. The polynucleotides in (ii) and (iii) above may be obtained by modifying the polynucleotide in (i) above using a known genetic engineering technique, and also be obtained by chemical synthesis.

The polynucleotides in (i) to (iii) above are used by being ligated to the side of 5′ end of a polynucleotide encoding a recombinant protein as 5′UTR in the production of the recombinant protein in a plant.

### (2) Nucleic Acid Construct Including the DNA Molecule

The nucleic acid construct of the present invention is characterized in that the polynucleotide in any of (i) to (iii) above is ligated to a polynucleotide encoding a protein.

In the nucleic acid construct of the present invention, the polynucleotide in any of (i) to (iii) above, which functions as 5′UTR, may just be ligated to the side of 5′ end of a polynucleotide encoding a protein.

In the nucleic acid construct of the present invention, the kind of the protein being encoded is not particularly limited, but may just be one of which production as a recombinant protein is demanded, such as a protein having pharmacological activity, for example. Specific examples of such a protein include enzymes, transcriptional factors, cytokine, membrane-bound proteins, various peptide hormones (e.g., insulin, growth hormone, and somatostatin), and medical proteins such as vaccines and antibodies. Also, in the nucleic acid construct of the present invention, a polynucleotide encoding a reporter protein such as GFP and luciferase and a tag peptide such as His tag and FLAG (registered trademark) tag may be ligated to the polynucleotide encoding a protein described above.

In the nucleic acid construct of the present invention, a known polynucleotide may be used as the polynucleotide encoding a protein. The base sequence of such a polynucleotide may be obtained from a database such as sequence database GenBank managed by National Center for Biotechnology Information (NCBI), for example. Based on such base sequence information, it is possible to isolate a polynucleotide encoding a protein from various living organisms by a usual method such as PCR, for example. The polynucleotide in question is also commercially available from various distributers in the form of a cDNA library, for example. It is therefore possible to purchase and use this.

In the nucleic acid construct of the present invention, the origin of the protein being encoded is not particularly limited, but the protein may just be a foreign protein homogeneous or heterogeneous to the host to be introduced into. In the nucleic acid construct of the present invention, if the codon usage of the host to be introduced into is known, the base sequence of the polynucleotide encoding a protein may be changed to correspond to the code usage suitable for the host in question.

### (3) Vector Including the Nucleic Acid Construct

The vector (expression vector) of the present invention is obtainable by linking the nucleic acid construct described above to a vector so as to be expressible. More specifically, the vector of the present invention may be obtained by linking the above nucleic acid construct to a vector provided with a promotor sequence at a position immediately after the transcription initiation site of the promotor.

The vector into which the nucleic acid construct is to be inserted is not particularly limited as long as it is replicable inside the host. Examples of such a vector include plasmid vectors, cosmid vectors, virus vectors, and artificial chromosomal vectors (e.g., YAC, BAC, and PAC). Among others, plasmid vectors and virus vectors are preferable. In particular, from the standpoint of expressing recombinant proteins in plants (including plant cells) further efficiently, an agrobacterium-derived plasmid is more preferable, and an agrobacterium-derived plasmid having T-DNA (Ti-plasmid) is especially preferable.

According to the present invention, a vector having a promotor sequence is used. As the promotor sequence, appropriate one may be selected according to the kind of the host. For example, CaMV35S promotor that is a promotor derived from cauliflower mosaic virus may be used.

The vector into which the nucleic acid construct is to be inserted may include a gene usable as a selection marker, such as a drug-resistant gene.

As the vector into which the nucleic acid construct is to be inserted, known ones and ones commercially available from various distributers may be used.

The nucleic acid construct may be incorporated into and linked to the vector according to a known genetic engineering technology. For example, the nucleic acid construct may be amplified by a PCR method using a restriction enzyme site-added primer, treated with a restriction enzyme, and linked to a restriction enzyme-treated vector.

While the vector of the present invention has the above nucleic acid construct linked immediately after the transcription initiation site of the promotor, a restriction enzyme site is to be present in a linkage portion between the promotor sequence and the nucleic acid construct according to the above cloning technique using the restriction enzyme, for example. In such a case, inverse PCR may be performed to remove the restriction enzyme site, and the resultant amplified product may be subjected to self-ligation, to prepare a vector excluding the restriction enzyme site present in the linkage portion. In this case, the primer set used for the inverse PCR is preferably designed to allow the PCR amplified product to be subjected to self-ligation. A ligase may be used for the self-ligation.

The wording of linking the above nucleic acid construct “immediately after the transcription initiation site” of the promotor sequence refers to linking the nucleic acid construct and the promotor sequence so as to obtain a transcript in which a base transcribed from the promotor sequence having 0, 1, 2, or 3 bases (preferably, 0, 1, or 2 bases) is coupled to 5′ end of produced mRNA (i.e., 5′UTR end) inside the host at the time when the polynucleotide encoding a protein is transcribed in the nucleic acid construct. In other words, they are linked so that no extra base sequence is present between the promotor sequence and the nucleic acid construct. Even in such direct linking between the promotor sequence and the nucleic acid construct, a few bases (e.g., 1, 2, or 3 bases) of the promotor sequence may be transcribed at the time of gene expression in some cases, and a vector causing such transcription is also included in the vector of the present invention.

### (4) Transformant Including the Vector

The transformant of the present invention is obtainable by introducing the vector of the present invention into a plant or a plant cell.

The kind of the plant to be used as the host is not particularly limited, but dicotyledoneae may be used, for example. More specific examples include Arabidopsis, tobacco, soybean, chrysanthemum, and lettuce.

The kind of the plant cell to be used as the host is not particularly limited, but dicotyledoneae-derived cells may be used, for example. More specific examples include Arabidopsis-derived cells, tobacco-derived cells, soybean-derived cells, chrysanthemum-derived cells, and lettuce-derived cells. Protoplasts derived from plant cells are also included in the plant cells. Plants obtained by culturing transformed plant cells are also included in the transformant of the present invention.

If a tumor tissue, a shoot, or a hairy root is obtained as a result of transformation, it can be used for cell culture, tissue culture or organ culture as it is. Also, it can be used to regenerate a plant by administering an appropriate concentration of a plant hormone such as auxin, cytokinin, gibberellin, abscisic acid, ethylene, and brassinolide, for example, using a conventionally known plant tissue culture method. It is also possible to regenerate a transformed plant using a transformed plant cell. As a regeneration method, employed is a method in which a callus-like transformed cell is moved to a medium with a hormone of a different kind and concentration and cultured, to form a somatic embryo, thereby obtaining a complete plant. As the medium used, LS medium and MS medium may be used.

The method for introducing the above vector into a host is not particularly limited, and an appropriate known method may be selected according to the kinds of the host and vector. Examples of such a method include an electroporation method, a particle gun method, and a method using Ti plasmid (e.g., a binary vector method and a leaf disk method).

Confirmation on whether the vector has been incorporated into a host can be performed by a PCR method, a southern hybridization method, a northern hybridization method, etc. For example, a DNA may be prepared from the transformant, and a vector-specific primer is designed, to perform PCR. Thereafter, the amplified product is subjected to agarose gel electrophoresis, polyacrylamide gel electrophoresis, capillary electrophoresis, or the like, and dyed with ethidium bromide, SYBR Green liquid, etc. The transformation is confirmed by detecting the amplified product as one band. The amplified product may also be detected by performing PCR using a primer previously labeled with fluorescent dye, etc. It is also possible to employ a method in which the amplified product is coupled to a solid phase such as a microplate, to confirm the amplified product with fluorescent light, enzyme reaction, or the like.

In the transformant of the present invention, transformed with the above vector, transcription of the mRNA and translation of the protein are performed from the polynucleotide encoding a protein. As described earlier, by using any of the polynucleotides (i) to (iii) above as 5′UTR, efficient expression of a recombinant protein is possible in a plant or a plant cell. Therefore, by culturing or cultivating the transformant of the present invention, it is possible to produce a recombinant protein efficiently. After the culture or cultivation of the transformant of the present invention, the recombinant protein may be collected and purified by a known technique, to obtain the recombinant protein.

### EXAMPLES

While the present invention will be described hereinafter in a specific manner, it should be noted that the present invention is not limited to the following examples. First, materials used for experiments will be described, followed by specific experiment details and results.

### 1. Used Plant and Cultured Cells

The following plant and cultured cells were used for examinations below.

### 1-1. Arabidopsis Plant

Seeds of Arabidopsis (Arabidopsis thaliana Columbia-0 (Col-0)) were sterilized with a solution of 5% sodium hypochlorite and 0.05% Triton-X for 10 minutes, then rinsed with sterile distilled water, and vemalized in a 4° C. dark place in a refrigerator (MPR-514, Panasonic, Osaka, Japan) for 2 days. The vernalized seeds were then planted in GM medium, and the day of transition to the growing condition was decided as day 0 of germination. The seeds were nourished in a growth chamber (BIOTRON, NK system, Osaka, Japan) that was under a condition of 16-hour light period/8-hour dark period at 22° C.

### 1-2. Arabidopsis T-87 Cultured Cells

As Arabidopsis cultured cells (Arabidopsis thaliana T87), those supplied from RIKEN Gene Bank, Plant Cell Bank were used. Culture was performed under conditions of 22° C., 24-hour light period, and a shaking rate of 80 rpm (SLK-3-FS, NK system), and 95 mL of modified LS medium (Nagata, T., Nemoto, Y, Hasezawa, S (1992), Tobacco BY-2 cell line as the HeLa cell in the cell biology of higher plants, Int. Rev. Cytol., 132, 1-30) placed in a 300 mL conical flask was used. Cells, 8 mL, having reached the stationary phase were transplanted to a new medium, 95 mL, every week and subcultured.

### 2. Tobacco Plant

As tobacco (Nicotiana benthamiana), one provided by National Institute of Advanced Industrial Science and Technology, Hokkaido was used. Sterilization was performed in the same manner as that for Arabidopsis plant except that the sterilization time was 30 minutes. The method of vemalization was also the same as that for Arabidopsis plant. After the vernalization, seeds were planted in a pot with soil (culture soil, Nihon Hiryo Co., Ltd., Tokyo, Japan) inside, germinated, and nourished in a greenhouse. On day 14 of germination, each plant was transplanted to a new pot and grown.

### 3. Experiment Details and Results

3-1. Polysome/Microarray Analysis as Evaluation Method of Translational State of mRNA

In general, the translational state of mRNA is determined using the number of ribosomes coupled to the mRNA as an indicator, such as that translation is active when a number of ribosomes are coupled to the mRNA (polysome) and no translation is underway when no ribosome is coupled (nonpolysome) (Bailey-Serres, J., Sorenson, R., Juntawong, O. (2009), Getting the message across: cytoplasmic ribonucleoprotein complex, Trends Plant Sci., 14:443-453). Such a technique is called polysome analysis, which makes it possible to evaluate the translational state of mRNA in a genome scale in combination with DNA microarray analysis.

### 3-2. PR Value as Indicator of Translational State

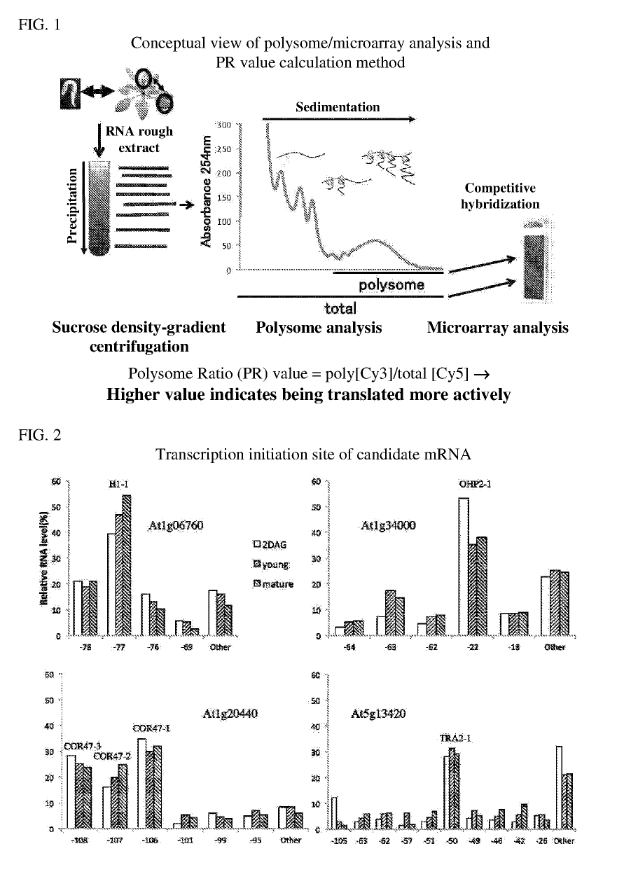

The polysome ratio (PR) value is a digitized value of the ratio of the quantity in polysome fractions to the total mRNA quantity in each mRNA, which is one of indicators representing the translational state. As the PR value of mRNA is higher, the mRNA is assumed to be actively translated. This being the case, samples were prepared from Arabidopsis plants different in growing stage and developmental stage, and subjected to polysome/microarray analysis, to calculate the PR values of all mRNA species in each sample. The conceptual view of this is shown in FIG. 1.

### 3-3. Polysome Analysis Using Sucrose Density-Gradient Centrifugation

Polysome analysis using sucrose density-gradient centrifugation was performed basically according to the method by Davies et al. except for a few modifications made to the method (Davies, E. and Abe, S. (1995), Methods for isolation and analysis of polyribosomes, Methods Cell Biol., 50, 209-222). As samples at different growing stages, all plants on day 2 of germination (seedlings) and day 21 (grown plants), and as samples at different developmental stages, the first to third leaves, the first leaf being the youngest leaf on day 21 of germination, as young leaves (expanding leaves), and the sixth to eighth leaves as mature leaves (expanded leaves) were cut off with scissors, put into a mortar with liquid nitrogen inside, crushed, and then stored at −80° C. Roughly the double amount (w/v) of an extraction buffer (200 mM Tris-HCl, pH 8.5, 50 mM KCl, 25 mM MgCl₂, 2 mM EGTA, 100 μg/mL heparin, 100 μg/mL cycloheximide, 2% polyoxyethylene 0-tridecyl ether, and 1% sodium deoxycholate) was added to the sample crushed powder, and the powder was mildly suspended. The suspension was centrifuged (14,000×g, 15 min., 4° C.) to remove cell residues, and further centrifuged (14,000×g, 10 min., 4° C.), to take its supernatant as a RNA rough extract. This rough extract was adjusted to a RNA concentration of 200 ng/μL to 800 ng/μL with the extraction buffer, and 300 μL of the extract was layered on 4.85 mL of a pre-prepared 26.25 to 71.25% sucrose density-gradient liquid (sucrose, 200 mM Tris-HCl, 200 mM KCl, 200 mM MgCl₂), and subjected to ultracentrifugation (SW55Ti rotor, 55,000 rpm, 50 min., 4° C., brake-off) (Optima. Beckman Coulter, California, USA). The absorbance at 254 nm was recorded using Bio-Mini UV Monitor AC-5200 (ATTO, Tokyo, Japan) simultaneously with aspiration at a speed of approximately 1 mL/min. from an upper portion of the sucrose density-gradient with Piston Gradient Fractionator (BioComp, Churchill Row, Canada).

### 3-4. Extraction of RNA for Microarray Analysis

The sucrose density-gradient liquid after the ultracentrifugation was divided into 8 fractions, and polysome RNA and total RNA were extracted from a polysome fragment that is a mixture of the first to third fragments (the first fragment being on the bottom side) and a total fragment that is a mixture of the first to eighth fragments. Each fragment was collected into a tube to which 8M guanidinium hydrochloride was previously added to obtain a final concentration of 5.5 M. At this time, with respect to spike mix A and spike mix B included in Two-Color RNA Spike-In Kit (Agilent Technologies, USA), spike mix A was added to the polysome fragment, and spike mix B was added to the total fragment. In each spike mix, 10 kinds of transcripts having a poly-A sequence synthesized in vitro are mixed in a 200-time dynamic range and at a known quantity ratio. Spots corresponding to these transcripts are present in Agilent oligoarray (Arabidopsis 3 oligo microarray 44K; Agilent Technologies) used in this study. Since RNA spike-in has been added simultaneously with collection of the sucrose density-gradient centrifugation liquid, it is to undergo subsequent processes such as RNA purification, labeling, and hybridization (to be described later). Therefore, by performing correction using signal values of spots corresponding to RNA spike-in, it becomes possible to estimate the actual RNA ratio (polysome RNA vs. total RNA) in the sucrose density-gradient (Melamed, D. and Arava, Y. (2007), Genome-wide analysis of mRNA polysomal profiles with spotted DNA microarrays, Methods Enzymol., 431:177-201). An equal quantity of 100% ethanol was added to the mixed liquid of the sucrose solution and guanidinium hydrochloride, and the resultant mixture was cooled overnight at −20° C. and then subjected to centrifugal operation (20,000×g, 45 min., 4° C.). The obtained pellet was washed once with 85% ethanol, then dissolved in a buffer RLT included in RNeasy kit (Qiagen, Germany), and thereafter subjected to RNA purification using RNeasy kit according to the attached protocol. Thereafter, purification with LiCl precipitation was performed. The quality of RNA was assayed by an on-chip electrophoresis method using Agilent Bioanalyzer 2100 (Agilent Technologies).

### 3-5. Microarray Hybridization

Complementary RNA (cRNA) fluorescently labeled with cyanine3 (Cy3) and cyanine5 (Cy5) was prepared from polysome RNA and total RNA, respectively, derived from the same sucrose density-gradient. The prepared cRNA was then subjected to a competitive hybridization experiment using Agilent oligoarray (Arabidopsis 3 oligo microarray 44K). In the Arabidopsis 3 oligo microarray, 60 mer oligo DNA, selected from base sequences of Arabidopsis-derived transcripts, the above-described RNA spike-in, etc., was printed at 44000 spots. For RA amplification and fluorescent labeling, Low RNA Input Fluorescent Liner Amplification Kit (Agilent Technologies) was used. First, using 500 ng of RNA as a template, reverse transcription reaction was performed using an oligo dT primer including a T7 promotor sequence as a linker sequence and MMLV-RT. Using the synthesized cDNA as a template, cRNA incorporating CTP labeled with Cy3 (polysome RNA) or Cy5 (total RNA) was synthesized by T7 RNA polymerase in vitro transcription reaction. The synthesized cRNAs were purified using RNeasy kit. The cRNAs, 750 ng each, were mixed together and subjected to 65° C. 17-hour hybridization reaction. After washing of the slide, scanning was performed using Agilent Technologies Microarray Scanner (Agilent Technologies), to detect signals of Cy3 and Cy5.

### 3-6. Microarray Data Analysis

Data was extracted from scanned images using Feature extraction software (Agilent Technologies). Based on flags set according to the setting criteria of the Feature extraction software, the following spots for either Cy3 or Cy5 were excluded from the subsequent analyses: spots in which signal values are saturated (glsSaturated, rlsSaturated), spots in which signals are not uniform (glsFeatNonUnifOL, rlsFeatNonUnifOL), spots in which gene corresponding to multiple spots is outlier (glsFeatPopnOL, rlsFeatPopnOL), and spots in which signals and background have no superiority (glsPosAndSignif, rlsPosAndSignif) (glsWellAboveBG, rlsWellAboveBG). Normalization was performed using a method based on spots corresponding to the RNA spike-in, or a Linear & LOWESS (Locally Weighted Linear Regression) method that is a standard normalization method in Feature extraction software. For the spots remaining as analytical objects, the PR value, polysome ratio=Cy3 (polysome RNA) signal value/Cy5 (total RNA) signal value, was calculated as the indicator of the translational state.

3-7. Selection of Candidate mRNA Having High PR Value for Each Sample

Using polysome RNA and total RNA prepared from day 2 of germination (seedlings), day 21 (grown plants), and young leaves (expanding leaves) and mature leaves (expanded leaves) on day 21 of germination, the PR values of 16348 mRNA species were calculated, and the following 4 species exhibiting high PR values in all samples were selected as candidate mRNA (each PR value is shown in parentheses, which is the ratio of polysome RNA that is being actively translated to total RNA).

(I) At1g06760 (Histone H1: H1): day 2 (0.62), day 21 (0.55), young leaf (expanding leaf) (0.56), mature leaf(expanded leaf) (0.58)

(II) At1g34000 (One-Helix Protein 2: OHP2): day 2 (0.73), day 21 (0.66), young leaf (expanding leaf) (0.64), mature leaf (expanded leaf) (0.67)

(III) At1g20440 (Cold-Regulated 47: COR47): day 2 (0.79), day 21 (0.69), young leaf (expanding leaf) (0.69), mature leaf (expanded leaf) (0.76)

(IV) At5g13420 (Transaldolase 2: TRA2): day 2 (0.71), day 21 (0.67), young leaf (expanding leaf) (0.65), mature leaf (expanded leaf) (0.67)

The PR values of average genes were as follows: At3g18780 (Actin 2), day 2 (0.60), day 21 (0.31), young leaf(expanding leaf) (0.33), mature leaf(expanded leaf) (0.27); and At3g47610, day 2 (0.59), day 21 (0.31), young leaf(expanding leaf) (0.35), mature leaf (expanded leaf) (0.32), both indicating that, while comparatively high PR values were shown on day 2 of germination, they were low in grown/developed tissues, exhibiting bad translational state.

### 3-8. Identification of Transcription Initiation Site by CAGE Analysis

For translation efficiency, 5′UTR plays an important role. It is therefore expected for 5′UTR of candidate mRNA to contribute to high translation efficiency. However, in some mRNA species, a plurality of transcription initiation sites (a plurality of mRNAs different in 5′UTR) are present. So, in order to determine the sequence of 5′UTR of candidate mRNA, information from data using a CAGE analysis method capable of determining the transcription initiation site in a genome-wide manner was extracted. A CAGE library was produced according to the technique developed by Kodzius et al. (Kodzius, R., Kojima, M., Nishiyori, H., Nakamura, M., Fukuda, S., Tagami, M., Sasaki, D., Imamura, K., Kai, C., Harbers, M., Hayashizaki, Y., Caminci, P. (2006), CAGE: cap analysis of gene expression, Nat. Methods, 3:211-22). First, with 5 μg of total RNA prepared using TRIzol (Invitrogen) from Arabidopsis plant as a template, reverse transcription reaction using N15 random primer and Primer Script Reverse Transcriptase (Takara) was performed, to synthesize cDNA. For the synthesized RNA/cDNA complex, cDNA was purified using Agencourt RNAClean XP (Beckman). CAGE linker was then coupled to the cDNA. The CAGE linker is a known ssDNA and includes MmeI and XmaJI restriction enzyme sites. Also, Biotin is added to 5′ end of ssDNA. After the coupling, dsDNA was synthesized from ssDNA using the CAGE linker as a primer. The synthesized dsDNA was subjected to MmeI restriction enzyme treatment. MmeI, which is a class I restriction enzyme, cleaves 20 bases downstream from 5′ end of the recognition site. In this experiment system, the cDNA-derived region coupled to the CAGE linker corresponds to the cleavage site. A known linker was further coupled to the MmeI cleavage site. At this stage, dsDNA (CAGE tag) with known sequences added at both ends, which included a sequence corresponding to 20 bases at 5′ end of mRNA was obtained. Further, an extra linker region was removed by treatment with restriction enzyme XmaJI, and CAGE tags were linked together, to obtain a CAGE library. Sequence analysis was performed using illumine(R) HiSeq 2000 according to the attached protocol. The CAGE linker sequence was removed from the obtained raw data, and tags corresponding to error read and liposomal RNA were removed. Mapping was then performed based on information of TAIR 10 (http://www.arabidopsis.org). The position of 5′ end of each tag on a chromosome was used as the transcription initiation site, and the number of tags at each transcription initiation site was counted. However, since cap-derived G was added to 5′ end of mapped tag sequence, the position after removal of G was determined as the actual transcription initiation site. Thereafter, only the transcription initiation site at which tags were present in both of analyzed two repetitions of data was selected, and the number of tags was converted to a tag per million (TPM) value. Annotation of each transcription initiation site was performed based on the information of TAIR 10: annotation was provided for a transcription initiation site included in the range from 500 upstream of the transcription initiation site described in TAIR 10 to CDS region for each gene. For each gene, then, the total value of TPM values as the expression level and the distribution ratio of each transcription initiation site were calculated.

FIG. 2 shows the transcription initiation site and distribution ratio of each candidate mRNA (the horizontal axis of the graph represents the number of bases from initiation codon AUG, and the vertical axis represents the relative ratio of each transcription initiation site to the total number of tags). From the results, sequences from major transcription initiation sites of each candidate mRNA were narrowed down, and selected ones were used as candidate 5′UTR for subsequent experiments. One having a plurality of major transcription initiation sites, if any, was also selected as a candidate. Detailed sequences are shown in Table 1, in which 5′UTR of At1g06760 was abbreviated as H1-1, 5′UTR of At1g34000 as OHP2-1, 5′UTRs of At1g20440 as COR47-1, COR47-2, and COR47-3, and 5′UTR of At5g13420 as TRA-1.

TABLE 1

| | | | Total number |
|---|---|---|---|
| AGI code | Abbr | Base sequence | of bases |
| At1g06760 | H1-1 | 5′-CAATCCTCATAATCACTTTCGAAATTACATTTACGCTTTCTTGCAATCAAATTTTCCGATCTTA | 77 |
| | | AGTTCAGAAGACG-3′ (SEQ ID NO: 2) | |
| At1g34000 | OHP2-1 | 5′-AGACAATTCAACTAACAAAAAA-3′ (SEQ ID NO: 7) | 22 |
| At1g20440 | COR47-1 | 5′-AAACATTACTCATTCACAAAACCATCTTAAAGCAACTACACAAGTCTTGAAATTTTCTATATTT | 106 |
| | | TCTATTTACTATATAAACTTTTAATCAAATCAAGATTAACT-3′ (SEQ ID NO: 1) | |
| | COR47-2 | 5′-CAAACATTACTCATTCACAAAACCATCTTAAAGCAACTACACAAGTCTTGAAATTTTCTCATAT | 107 |
| | | TTTCTATTTACTATATAAACTTTTAATCAAATCAAGATTAACT-3′ (SEQ ID NO: 2) | |
| | COR47-3 | 5′-ACAAACATTACTCATTCACAAAACCATCTTAAAGCAACTACACAAGTCTTGAAATTTTCTCATA | 108 |
| | | TTTTCTATTTACTATATAAACTTTTAATCAAATCAAGATTAACT-3′ (SEQ ID NO: 3) | |
| At5g13420 | TRA-1 | 5′-GATCGATCAAACCAAGAAAAAACACTTCGTATTTCCCTCGACGAAAAAA-3′ | 50 |
| | | (SEQ ID NO: 8) | |
| At1g77120 | ADH | 5′-ACATCACAATCACACAAAACTAACAAAAGATCAAAAGCAAGTTCTTCACTGTTGATA-3′ | 57 |
| (control) | | (SEQ ID NO: 9) | |
| Vector- | | 5′-CACGGGGGACTCTAGAAGATCCCCGGGTAGGTCAGTCCCTT-3′ | |
| derived | | (SEQ ID NO: 10) | |
| (control) | | | |

### 3-9. Evaluation of Translational Ability of Candidate 5′UTR by DNA Transient Expression Experiment

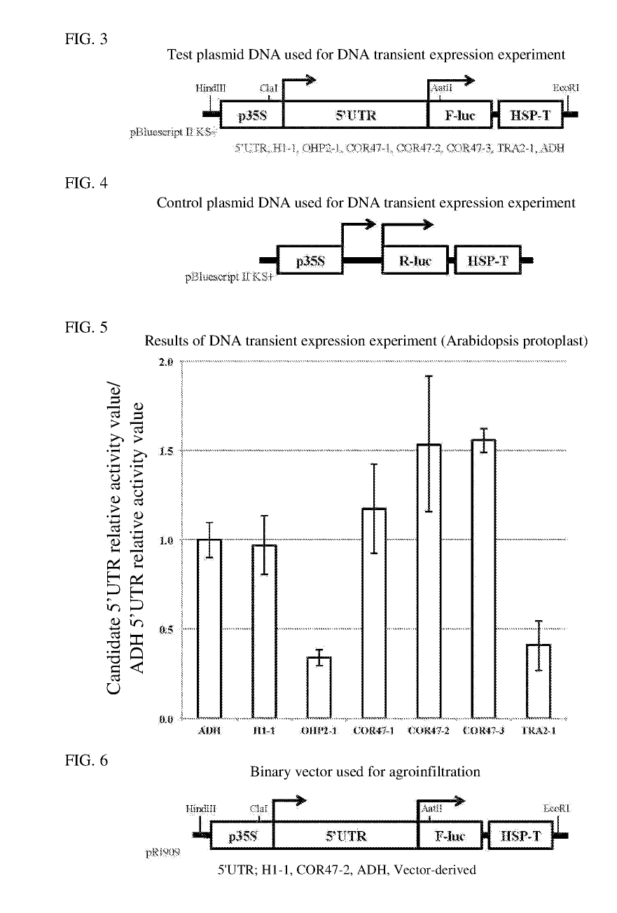

In order to evaluate the translational ability of the obtained candidate 5′UTR (total 6 species), a DNA transient expression experiment was performed using protoplast prepared from Arabidopsis cultured cells. The 5′UTR to be tested was amplified by PCR using Arabidopsis genome DNA as a template with each 5′UTR-specific primer having ClaI site at 5′ end and AatII site at 3′ end, and inserted into HincII site of pUC118 vector. It was then cleaved at ClaI and AatII, inserted into ClaI/AatII site of plasmid pBluescriptIIKS+ having F-luc gene and HSP terminator under the control of 35S promotor, to obtain test plasmid DNA (FIG. 3, in which the arrows indicate the transcription initiation site and translation initiation site). For R-luc gene for correction, pBluescriptIIKS+ having an expression cassette made of 35S promotor, R-luc gene, and HSP terminator was used (FIG. 4). DNA (F-luc 0.4 μg, R-luc 0.04 μg, total volume approx. 5 μl) was introduced into protoplast prepared from Arabidopsis T87 cultured cells by a polyethylene glycol (PEG) method (Kovtun, Y., Chiu, W. L., Tena, G., Sheen, J. (2000), Functional analysis of oxidative stress-activated mitogen-activated protein kinase cascade in plants, Proc. Natl. Acad. Sci. USA, 6:2940-2945), left to stand at 22° C. for 6 hours, and then centrifuged to remove supernatant. It was then frozen with liquid nitrogen and stored at −80° C. Thereafter, cells were dissolved using a passive lysis buffer (Promega Wisconsin, USA), and F-luc and R-luc activities in the solution were measured with Dual-luciferase reporter assay system (Promega) and a luminometer (Lumat LB 9501. Berthold, Northern Black Forest, Germany), to determine the relative activity value (F-luc activity value/R-luc activity value). Similar evaluation was also made for 5′UTR of At1g77120 (alcohol dehydrogenase: ADH) already known as having high translational ability (Sugio, T., Satoh, J., Matsuura, H., Shinmyo, A., Kato, K. (2008), The 5′-untranslated region of the Oryza sativa alcohol dehydrogenase gene functions as a translational enhancer in monocotyledonous plant cells, J. Biosci. Bioeng., 105:300-302) (Table 1), and the relative activity value of each candidate 5′UTR was calculated as the relative activity value with respect to the 5′UTR of ADH (relative activity value of candidate 5′UTR/relative activity value of 5′UTR of ADH) (FIG. 5).

As a result, it has been clarified that four 5′UTRs of H1-1, COR47-1, COR47-2, and COR47-3 exhibit translational ability equivalent or superior to the 5′UTR of ADH, contributing to improvement of the production efficiency of recombinant proteins in plants.

Since the base sequences of COR47-1, COR47-2, and COR47-3 are only different in one base or two bases, H1-1 and COR47-2 were tested as candidate 5′UTR in the subsequent experiments.

### 3-10. Evaluation of Translational Ability of Candidate 5′UTR by Agroinfiltration

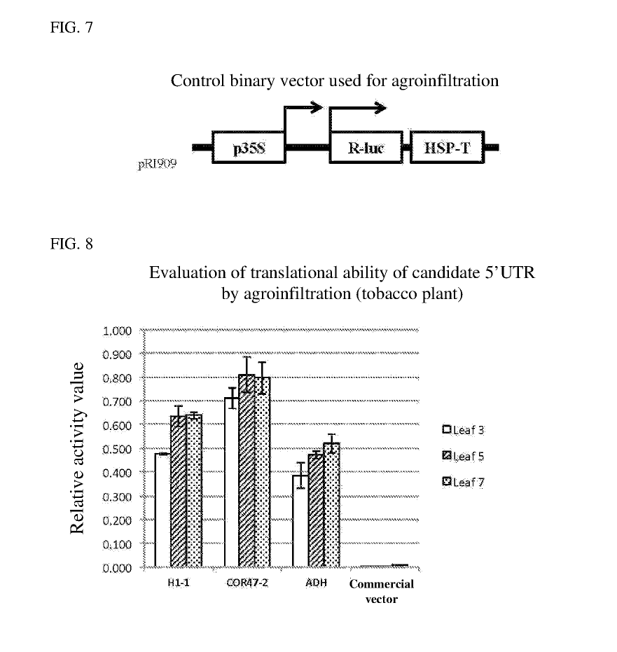

In order to evaluate the expressional ability of candidate 5′UTR for plants, agroinfiltration using tobacco (Nicotiana benthamiana) was performed. The plasmid DNA (H1-1, COR47-2, ADH) used for the DNA transient expression experiment was treated with restriction enzymes HindIII/EcoRI/PvuII, to cleave out a portion of p35S::5′UTR::F-luc::tHSP. This fragment was inserted into HindIII/EcoRI site of binary vector pRI909 (Takara), to construct a binary vector (FIG. 6). A binary vector was also constructed in a similar manner for a generally used commercial expression cassette (p35S::vector-derived 5′UTR::F-luc::tNOS) (FIG. 6). Table 1 shows the base sequence of the vector-derived 5′UTR. For an expression cassette having R-luc gene for correction, also, a binary vector was constructed in a similar manner (FIG. 7). Agrobacterium retaining each of these binary vectors was cultured in 5 ml of 2×YT medium at 28° C. for 30 hours, then centrifuged at 5,000 rpm (MX-300, Tomy Seiko, Tokyo, Japan), and collected in a 50 ml Falcon tube. The pellet was suspended in an infiltration buffer (10 mM MgCl₂, 10 mM MES-KOH pH5.8, 100 μM Acetosyringone) and adjusted so that the value of O.D.₆₀₀ was 1 with a spectrophotometer. Agrobacterium retaining the binary vector having F-luc and agrobacterium retaining the binary vector having R-luc were mixed at a ratio of 9:1, and left to stand at room temperature for 3 hours. The resultant agrobacterium, 50 μl each, was poured to the third, fifth, and seventh leaves, with the first being the youngest leaf, of tobacco (Nicotiana benthamiana) nourished in a greenhouse for 40 days, using a 1 ml medium syringe (Terumo, Tokyo, Japan). One zirconia bead (BMS, Tokyo, Japan) was put in each of 2 ml tubes, and each 3 punches of leaf fragments taken from the third, fifth, and seventh leaves 3 days after the introduction of agrobacterium using Biopsy Punch (Kai Industries, Gifu, Japan) were sampled and put into the tubes, and frozen with liquid nitrogen. The samples were then crushed with TissueLyser II (QIAGEN) at a frequency of 20/s for 30 seconds, and dissolved in an added passive lysis buffer with a mixer at room temperature for 10 minutes. The luciferase activity was then measured, to calculate the relative activity value (F-luc activity value R-luc activity value) (FIG. 8).

As a result, H1-1 and COR47-2 as candidate 5′UTR exhibited high relative activity values in each leaf compared with 5′UTR of ADH. By contrast, the commercial expression cassette exhibited only very low relative activity values, as low as 1/180 of those of COR47-2.

### 3-11. Improvement of Translational Ability by Modification of Initiation Codon-Neighboring Sequence of Candidate 5′UTR

While 5′UTR is a very important factor that defines the translation efficiency of mRNA, it is known that an initiation codon-neighboring sequence in the 5′UTR also affects the translation efficiency (Sugio, T., Matsuura, H., Matsui, T., Matsunaga, M., Nosho, T., Kanaya, S., Shinmyo, A., Kato, K. (2010), Effect of the Sequence Context of the AUG Initiation Codon on the Rate of Translation in Dicotyledonous and Monocotyledonous Plant Cells, J. Biosci. Bioeng., 109:170-173). In view of this, an initiation codon-neighboring sequence of each of H1-1 and COR47-2 was substituted by a base considered better in efficiency, and a test plasmid similar to that in FIG. 3 was produced using the modified sequence and subjected to the DNA transient expression experiment using protoplast adjusted from Arabidopsis cultured cells. The base sequences of the modified 5′UTRs, named H1-1 mod and COR47-2 mod, are shown in Table 2. By a method similar to that for the previous experiment, the F-luc and R-luc activities were measured, and the relative activity value (F-luc activity value/R-luc activity value) was determined, to calculate the relative activity value of each modified 5′UTR with respect to the relative activity value of 5′UTR before modification as 1.

TABLE 2

| | | | Total number |
|---|---|---|---|
| AGI code | Abbr | Base sequence | of bases |
| At1g06760 | H1-1mod | 5′-CAATCCTCATAATCACTTTCGAAATTACATTTACGCTTTCTTGCAATCAAATTTTCC | 77 |
| | | GATCTTAAGTTCAGAAGAAG-3′ (SEQ ID NO: 6) | |
| At1g20440 | COR47- | 5′-CAAACATTACTCATTGACAAAACCATCTTAAAGGAACTAGACAAGTCTTGAAATTTT | 107 |
| | 2mod | CTCATATTTTCTATTTACTATATAAACTTTTAATCAAATCAAGATTAAAG-3′ | |
| | | (SEQ ID NO: 5) | |

As a result, the relative activity value of H1-1 mod was 1.03, and that of COR47-2 mod was 1.13, both exhibiting high activity values compared with the values before the modification.

### 3-12. Evaluation of Translational Ability of Candidate 5′UTR in Plant

In order to evaluate the ability of candidate 5′UTR (COR47-2) in a plant, a stable transformant was created. In this test, the binary vector constructed in “3-10. Evaluation of Translational Ability of Candidate 5′UTR by Agroinfiltration” (FIG. 6; COR47-2 was used as 5′UTR) described above was used. Further, for comparison, one in which COR-47-2 of 5′UTR of this binary vector was substituted by 5′UTR of At3g47610 mRNA (base sequence shown in Table 3) was constructed and used. Note that the 5′UTR of At3g47610 mRNA was confirmed to exhibit a low PR value in the grown/developed tissues, being low in translation efficiency in “3-7. Selection of Candidate mRNA having High PR Value for Each Sample” described above.

TABLE 1

| AGI code | 5′ 3′ | length |
|---|---|---|
| At3g47610 | GTCGTTTCGAAGAGACTAAAGGCGACGGAGAGAATCGGAGAAGAAG | 46 |

The above binary vector was introduced into Arabidopsis by a floral dip method using agrobacterium, to obtain T3 seeds. Samples of the created stable transformant were prepared at growing stages (day 2 and day 21 after germination) and developmental stages (young leaves and mature leaves on day 21 after germination), to perform polysome analysis. RNA was prepared from the entire plant for the growing stages, and RNA was prepared from only leaf portions excluding petioles for the developmental stages, and subjected to sucrose density-gradient centrifugation. The centrifuged liquid was then divided into 8 fractions (fractions 1 to 4 correspond to non-polysome (NP) fractions, and fractions 5 to 8 correspond to polysome (P) fractions). More specifically, approximately 500 μL of the sucrose density-gradient solution was collected into 8 tubes containing 5 ng of in vitro synthesized Renilla luciferase (R-luc) mRNA having a cap structure and a poly-A sequence and 8M guanidinium hydrochloride previously added so as to have a final concentration of 5.5 M. An amount of 100% ethanol equal to the mixed liquid in each tube was added, and the resultant liquid was cooled overnight at −20° C. and then centrifuged (14,000×rpm, 45 min., 4° C.). The obtained pellet was washed once with 85% ethanol, then dissolved in a buffer RLT included in RNeasy kit (Qiagen, Hilden, Germany), and thereafter subjected to RNA purification with RNeasy kit according to the attached protocol. The purified RNA solution divided in equal volumes was subjected to reverse transcription reaction. The reverse transcription reaction was performed with Transcription First Strand cDNA Synthesis Kit (Roche Applied Science, Penzberg, Germany) according to the attached protocol. The reaction liquid was 13 μL (use of oligo dT primer). Using 2 μL of a reverse transcription reaction solution diluted 5 to 40 times as a template, PCR was performed with 10 μL of the reaction liquid using a gene-specific primer set and LightCycler 480 SYBR Green I Master (Roche Applied Science). Universal ProbeLibrary Assay Design Center/ProbeFinder (Roche Applied Science) was used for design of the primer, LightCycler 480 System (Roche Applied Science) was used for measurement over time of the fluorescence intensity of SYBR Green I, and the second derivative maximum method of LightCycler Data Analysis Software (Roche Applied Science) was used for data analysis. In order to correct the difference in RNA collection efficiency and RT-PCR reaction efficiency among fragments, the result of the abundance of target mRNA in each fragment was corrected with the result of R-luc mRNA for correction added at the collection of the sucrose density-gradient liquid. That the signal was not derived from genome was confirmed by detection of no signal in PCR using a RNA solution that was not subjected to reverse transcription reaction as a template. The amount of F-luc mRNA ligating candidate 5′UTR and internal COR47 mRNA present in each fragment was quantified by qRT-PCR analysis, and calculated as a ratio to the total amount of F-luc mRNA ligating candidate 5′UTR and internal COR47 mRNA present in all fragments.

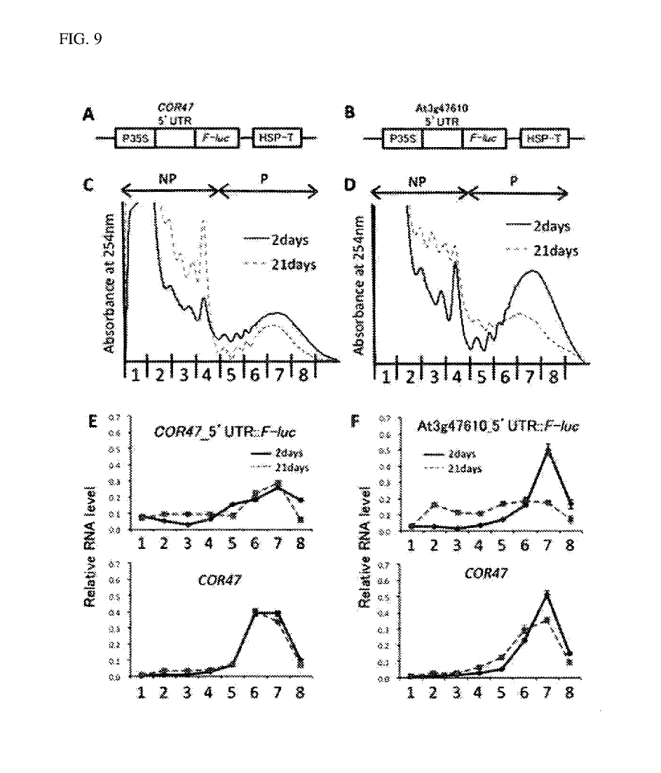

The results of polysome/qRT-PCR analysis at growing stages are shown in FIG. 9. In C to F of FIG. 9, the numbers on the horizontal axis refer to the divided fraction numbers. In E and F in FIG. 9, the vertical axis represents the relative value of the amount of mRNA present in each fragment with respect to the total amount of target mRNA present in all fragments as 1. While, the abundance of F-luc mRNA ligating 5′UTR of At3g47610 was very high in the polysome fragments for samples on day 2 of germination, it was high in the non-polysome fragments for samples on day 21 of germination, indicating that the translation state was bad. By contrast, as for F-luc mRNA ligating 5′UTR of COR47-2, many mRNAs were present in the polysome fragments both on day 2 and day 21 of germination, indicating that translation was active.

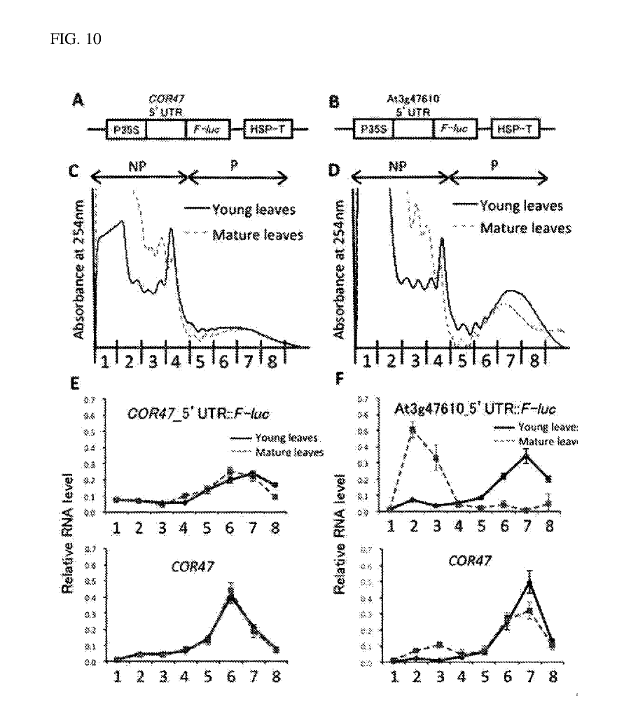

The results of polysome/qRT-PCR analysis at developmental stages are shown in FIG. 10. In C to F of FIG. 10, the numbers on the horizontal axis refer to the divided fraction numbers. In E and F in FIG. 10, the vertical axis represents the relative value of the amount of mRNA present in each fragment with respect to the total amount of target mRNA present in all fragments as 1. In these results, also, while, the abundance of F-luc mRNA ligating 5′UTR of At3g47610 was very high in the polysome fragments for samples of young leaves, the translation state was bad for samples of mature leaves. As for F-luc mRNA ligating 5′UTR of COR47-2, mRNAs were present in the polysome fragments both for young leaves and mature leaves, indicating that translation was active.

From the results described above, when 5′UTR of COR47-2 is ligated to the target gene, it is considered possible to translate the target protein efficiently even under the circumstances where the translation state as the entire cell is worsened with growth and development of the cell.

1. A DNA molecule encoding a 5′ UTR, having a polynucleotide sequence defined in any of (i) to (iii) below:
(i) a polynucleotide sequence consisting of a base sequence represented by SEQ ID NO: 1;
(ii) a polynucleotide sequence consisting of a base sequence in which one or several bases of the base sequence represented by SEQ ID NO: 1 are substituted, deleted, or added, the polynucleotide exhibiting 5′ UTR activity equivalent to that of the polynucleotide having the base sequence represented by SEQ ID NO: 1; and
(iii) a polynucleotide sequence hybridized with a DNA fragment consisting of a base sequence complementary to the base sequence represented by SEQ ID NO: 1 under a stringent condition, the polynucleotide exhibiting 5′UTR activity equivalent to that of the polynucleotide sequence having the base sequence represented by SEQ ID NO: 1.

2. The DNA molecule according to claim 1, having a polynucleotide sequence consisting of a base sequence represented by SEQ ID NO: 1, SEQ ID NO: 3 or SEQ ID NO: 4.

3. A nucleic acid construct comprising the DNA molecule according to claim 1 that is linked to a 5′ end side of a polynucleotide encoding a protein.

4. A vector including the nucleic acid construct according to claim 3.

5. A method for producing a transformant comprising introducing the vector according to claim 4 to a plant or a plant cell.

6. A transformant obtained by transforming a plant or a plant cell with the vector according to claim 4.

7. A method for producing a recombinant protein comprising culturing or cultivating the transformant according to claim 6.

8. The DNA molecule according to claim 1, comprising a polynucleotide sequence consisting of a base sequence represented by SEQ ID NO: 3 or SEQ ID NO: 4.

9. A nucleic acid construct comprising the DNA molecule according to claim 2 that is linked to a 5′ end side of a polynucleotide encoding a protein.

10. A nucleic acid construct comprising the DNA molecule according to claim 8 that is linked to a 5′ end side of a polynucleotide encoding a protein.

11. A vector including the nucleic acid construct according to claim 9.

12. A vector including the nucleic acid construct according to claim 10.

13. A method for producing a transformant comprising introducing the vector according to claim 11 to a plant or a plant cell.

14. A method for producing a transformant comprising introducing the vector according to claim 12 to a plant or a plant cell.

15. A transformant obtained by transforming a plant or a plant cell with the vector according to claim 11.

16. A transformant obtained by transforming a plant or a plant cell with the vector according to claim 12.

17. A method for producing a recombinant protein comprising culturing or cultivating the transformant according to claim 15.

18. A method for producing a recombinant protein comprising culturing or cultivating the transformant according to claim 16.


SEQUENCE LISTING (10 sequences)

SEQ ID NO: 1 - DNA, 106 bp, Arabidopsis thaliana
aaacattactcattcacaaaaccatcttaaagcaactacacaagtcttgaaattttctcatattttctatttactatataaacttttaatcaaatcaagattaact

SEQ ID NO: 2 - DNA, 77 bp, Arabidopsis thaliana
caatcctcataatcactttcgaaattacatttacgctttcttgcaatcaaattttccgatcttaagttcagaagacg

SEQ ID NO: 3 - DNA, 107 bp, Arabidopsis thaliana
caaacattactcattcacaaaaccatcttaaagcaactacacaagtcttgaaattttctcatattttctatttactatataaacttttaatcaaatcaagattaact

SEQ ID NO: 4 - DNA, 108 bp, Arabidopsis thaliana
acaaacattactcattcacaaaaccatcttaaagcaactacacaagtcttgaaattttctcatattttctatttactatataaacttttaatcaaatcaagattaact

SEQ ID NO: 5 - DNA, 107 bp, Arabidopsis thaliana
caaacattactcattcacaaaaccatcttaaagcaactacacaagtcttgaaattttctcatattttctatttactatataaacttttaatcaaatcaagattaaag

SEQ ID NO: 6 - DNA, 77 bp, Arabidopsis thaliana
caatcctcataatcactttcgaaattacatttacgctttcttgcaatcaaattttccgatcttaagttcagaagaag

SEQ ID NO: 7 - DNA, 22 bp, Arabidopsis thaliana
agacaattcaactaacaaaaaa

SEQ ID NO: 8 - DNA, 50 bp, Arabidopsis thaliana
gatcgatcaaaccaagaaaaaacactttcgtatttccctcgacgaaaaaa

SEQ ID NO: 9 - DNA, 57 bp, Oryza sativa
acatcacaatcacacaaaactaacaaaagatcaaaagcaagttcttcactgttgata

SEQ ID NO: 10 - DNA, 41 bp, Artificial Sequence
  DNA coding 5′UTR of p35S vector
cacgggggactctagaggatccccgggtaggtcagtccctt